(19) United States
(12) Patent Application Publication
(10) Pub. No.: US 2005/0249819 A1
(43) Pub. Date: Nov. 10, 2005

(54) Anti-microbial carrier systems

(76) Inventors: Bennett C. Hinnen, Golden, CO (US); Dennis McWherter, Thornton, CO (US)

Correspondence Address: MARSH FISCHMANN & BREYFOGLE LLP;Suite 411, 3151 South Vaughn Way, Aurora, CO, 80014, US

(21) Appl. No.: 11/122,997

(22) Filed: May 5, 2005

Related U.S. Application Data
(60) Provisional application No. 60/568,435, filed on May 5, 2004.

(51) Int. Cl. C12M 1/34; A61K 33/38

(52) U.S. Cl. 424/618; 435/287.1

(57) ABSTRACT

An improved carrier system is provided that includes a plurality of carriers and/or a transport surface having anti-microbial characteristics. In a pneumatic tube carrier system application, the internal surfaces of one or a plurality of selectively openable/closeable carriers may comprise an anti-microbial material The anti-microbial material may be mixed with a base material to form each carrier. Alternatively, the anti-microbial material may be mixed in a coating applied to a base carrier structure. The carrier contact surfaces of the pneumatic tubes and other system componentry may also comprise an anti-microbial material.

### CROSS-REFERENCE TO RELATED APPLICATIONS

This application claims priority to U.S. Provisional Application No. 60/568,435 entitled “Anti-Microbial Carrier Systems” and having a filing date of May 5, 2004, the contents of which are incorporated herein by reference in their entirety.

### FIELD OF THE INVENTION

The present invention relates to the reduction of microbes in carrier systems, and more particularly, to the reduction of bacteria, fungi, and other microbes in carrier systems that comprise one or a plurality of carriers for separately carrying contained material and a transport surface for guiding and/or supporting the carrier(s) during transfer of the carrier(s) from one location to another, wherein a reduction of secondary microbe transmission may be realized. The invention is particularly apt for pneumatic tube carrier systems for transferring sealably contained materials, e.g. pneumatic tube systems for transporting bodily fluids/tissue within a medical facility.

### BACKGROUND OF THE INVENTION

Carrier systems are utilized in a wide variety of settings to transfer materials from one location to another. Such systems are typically automated, wherein material is moved in a driven manner relative to and/or together with a transport surface from one location to another. In many applications, carrier systems are employed to transfer materials that are contained by separate carriers. For example, selectively openable/closeable carriers are being employed to transport various materials in automated carrier systems in medical facilities.

More particularly, pneumatic tube carrier systems are being utilized in medical facilities to transport patient-specific materials that are contained within openable/closeable carriers from one location to another (e.g. from pharmacy to nurse station; from operating room to laboratory, etc.). Of note, such contained materials may comprise body/biological materials removed from one or more patients, (e.g. urine, blood, spinal, tissue and other body materials), wherein such body materials are maintained in a sterile fashion within various vessels (e.g. vials, syringes, bags, etc.) that are supportably positioned within the operable/closeable carriers. In turn, such carriers are driven via pneumatic pressure within interconnected tubes that extend between various locations in a medical facility.

In such arrangements, leakage and other unintended spillage of body materials is of concern. For example, the spillage of body material samples within a given carrier may introduce undesired microbes onto internal surfaces of the carrier. In turn, such microbes may be transferred to other items located within the carrier. Such items may then spread the microbes upon removal from the carrier. Further, in the event of leakage of body materials from within the carrier, additional secondary microbe transmission concerns arise. As such, in current pneumatic tube systems, carrier leakage of body fluids may result in a partial or total shut-down of the system to allow maintenance personnel to disinfect the tube system componentry.

### SUMMARY OF THE INVENTION

In view of the foregoing, a broad objective of the present invention is to reduce the presence of microbes in material carrier systems, including in particular, pneumatic tube systems.

A related objective of the present invention is to reduce the secondary transmission of microbes from and within material carrier systems.

Another related objective of the present invention is to reduce exposure of material carrier system users to microbes present within the system.

The above objectives are satisfied and additional advantages realized by the present invention which, according to a first aspect, incorporates a non-toxic inorganic anti-microbial material into components of a material carrier system. Generally such a carrier system comprises a plurality of carriers for carrying contained material and a transport surface for guiding and/or supporting the carriers during transfer of the carriers from one location to another. An internal surface of one or more of the carriers and/or the transport surface comprises an inorganic anti-microbial material that reduces the presence and growth of, for example, bacteria, mold and fungus.

In one approach, the system may be provided so that the carriers move relative to a stationary transport surface, e.g. wherein driven carriers are supported and/or guided by a rail, track or tube extending between origination and destination locations. Alternatively, the system may be provided so that the carriers and transport surface move together from one location to another. In either case, the carriers are in direct contact with the transport surface.

The anti-microbial material comprising the internal surface of the carriers and/or transport surface may include any of a number of anti-microbial materials that contain anti-microbial metal ions as active agents. These metallic ions may include, without limitation, Ag, Au, Pt, Pd, Ir (i.e. the noble metals), Cu, Sn, Sb, Bi and Zn. To increase the rate of diffusion of anti-microbial ions the anti-microbial metal ions may be provided in a carrier material. Such carrier materials enhance the release of metallic ions to establish increased anti-microbial levels of ions for a given material.

In one arrangement for enhancing the release of metallic ions, anti-microbial metals are incorporated (e.g., blended) into carrier materials, which may allow for substantially continuous release of metallic ions. Generally, such carrier materials are inorganic non-metallic materials that have a porosity that allows for release of metal ions from within their structure. For instance, some ceramic materials may be blended with metals to generate a composite material that allows for the controlled release of ions over an extended period of time (e.g., years). Such ceramic materials may include, inter alia, porcelains, silicates (e.g. glasses) that may be blended with metals such that they may to carry and release metal ions (e.g., silver). In this regard, the anti-microbial material may comprise antimicrobial particles (e.g., carrier material and metal ions) that are selected to provide for the controlled release of the anti-microbial metal ions over an extended period of time. In use, the released anti-microbial metal ions migrate toward the surface of the structure of which the anti-microbial particles are disposed on or disposed within to reduce microbes that contact such surface(s), e.g. pursuant to unintended vessel spillage or breakage. In one embodiment, the anti-microbial particles comprise glass particles containing silver ions. In another embodiment the anti-microbial particles comprise zeolite particles containing silver ions.

In one approach, carriers components of an improved carrier system may be formed utilizing a polymeric resin, wherein the anti-microbial materials and/or particles (i.e., antimicrobial material and carrier material) are mixed with the resin during fabrication (e.g. prior to molding). Alternatively, a coating comprising the anti-microbial material/particles may be applied to one or more surfaces of a base structure defining the component(s). Components within the system that may utilize such anti-microbial materials include, without limitation, carriers, tubes, user stations, traffic control devices and transfer units. Generally, any contact surface that contacts the carries within the system may be formed of a polymeric resin including an anti-microbial material/particle within its matrix and/or be coated with an anti-microbial coating material.

The concentration of the antimicrobial material in the polymeric resin and/or in the coating may be any concentration that provides antimicrobial functionality to the surface of the component. Generally, concentrations of an antimicrobial material and its carrier of between about 0.25 percent and about 5 percent by weight of the resin and/or coating will provide the desired antimicrobial functionality. In one arrangement, concentrations between about 0.5 percent and about 1 percent are utilized. However, it will be appreciated that the concentration in polymeric materials and/or coatings may be dependent upon the type of antimicrobial material utilized. Further, the size of individual antimicrobial particles (i.e., antimicrobial material and its carrier) may be selected for mechanical purposes. Generally, smaller individual antimicrobial particles have a greater surface area to volume ratio that permits the individual particles to more effectively release antimicrobial metal ions. Typically, individual antimicrobial particles may have a size of less than about 40 microns, more preferably less than about 20 microns and most preferably less than about 10 microns. Further, individual antimicrobial particles of different sizes may be utilized to increase a packing density of the antimicrobial particles within a matrix (e.g., polymeric resin) when appropriate.

According to another aspect of the present invention, an improved carrier for use in pneumatic tube transport system is provided. The carrier comprises a containment vessel having a sidewall that defines an enclosed space. The carrier further includes an access to the enclosed space such that materials may be selectively placed within the enclosed space. In this regard, the carrier is selectively openable/closeable for carrying contained material(s), for example, between user stations in the system. An antimicrobial material is associated with at least the inside surface of the sidewall of the containment vessel to inhibit microbial growth on the sidewall. The carrier is designed for insertion into/removal from one or a plurality of interconnected tubes extending between the user stations. Further, the system may include one or more traffic control devices for ordering, storing and/or otherwise routing carriers through the system.

In conjunction with this aspect, at least a portion of the side wall of the carrier is preferably translucent, wherein the presence of an object(s) located therein is discernable at predetermined incident light levels (e.g. at least 0.01 Illuminance (Lux)). In this regard, it should be noted that one or more of the user stations in the system may be provided with a carrier port having a light source. More particularly, the carrier port of one or more user stations may be provided with a light source so that, upon positioning of a carrier into the carrier port (e.g. upon sending or receipt) light emanating from the light source will “back-light” the carrier to help a user observe the presence or non-presence of items therewithin. Further, the light source may be placed adjacent to carriers or mounted beneath the carriers. In the latter case, the light may be protected (e.g., from impact damage) by a member that allows light to pass through. For example, a perforated stainless steel member may be utilized, or, the light may be placed beneath a translucent member (e.g., window). Accordingly, these structures could also be coated with antimicrobial substances and/or be formed to absorb the energy of carrier landing (e.g., as pliable surfaces, slings etc.).

In one approach, the sidewall/body of the carrier may be formed from a polymeric resin, wherein the anti-microbial material is mixed with the resin prior to molding of the carrier. For example, a selectively openable/closeable pneumatic tube carrier sidewall may comprise a polycarbonate material (clear or colored/opaque) and anti-microbial material, wherein the anti-microbial material and its carrier if utilized comprises between about 0.25 percent and about 0.5 percent of the sidewall by weight. In one arrangement, the antimicrobial material and its carrier if utilized comprises between about 0.5 percent and about 1.0 percent of the sidewall by weight. When used with a carrier, the anti-microbial material and its carrier may comprise particles having a size of less than about 40 microns and more preferably a size of less than about 20 microns. Most preferably a particle size of 10 microns or less is utilized. Of note, when producing an at least partially translucent carrier, it has been found that decreasing the particle size of the anti-microbial material may yield carriers having an improved clarity/translucency. Further in this regard, each container body may have a wall thickness of between about 0.050 in. and 0.260 in. and preferably about 0.150 in.

In another approach, a coating containing an anti-microbial material may be provided on at least the inside of the carriers, wherein the anti-microbial material and its carrier, if utilized, comprises between about 0.25 percent and about 5.0 percent of the coating by weight, and preferably between about 0.05 percent and 1.0 percent of the coating by weight. Further, the coating may have a thickness of between about 0.004 in. and 0.020 in. and preferably at least about 0.010 in.

Additionally, the improved carrier may include at least one and preferably at least two spaced wear bands that extend around and/or along the carrier. In turn, such wear bands may each include a plurality of fibers that extend outwardly away from the carrier. Preferably, the wear bands have a packing density of fibers of at least about 4000 fibers per square inch and more preferably between about 10,000 and 100,000 fibers per square inch. The fibers will typically have an outside diameter of less than about 0.010 inches and more preferably have an outside diameter between about 0.003 inches and about 0.0005 inches. Such fibers may be fabricated from an anti-microbial material mixed with a resin material and/or be coated with an antimicrobial material.

The carrier may further include a latch on its outside surface for securing the first access in a closed position. Such a latch may be formed of metal and/or a polymeric material. The latch may be operable to, for example, secure a lid that is pivotally hinged on the sidewall. Accordingly, antimicrobial material may be incorporated into or on the latch.

According to another aspect of the present invention, an improved pneumatic carrier system is provided that utilizes an antimicrobial pneumatic tube. In this regard, at least a first pneumatic tube pneumatically interconnects first and second locations within the system. This first pneumatic tube includes inner surface and an outer surface, where the inner surface defines a bore sized to receive a pneumatic carrier. An inorganic antimicrobial material is associated with at least the inside surface of the first pneumatic tube to inhibit microbial growth on the inside surface of the tube.

In one approach, one or more of such pneumatic tubes may be fabricated from a polymeric resin, wherein the anti-microbial material is mixed with the resin prior to fabrication of the pneumatic tubes. For example, each tube section may be fabricated from a polycarbonate material and anti-microbial material, wherein the anti-microbial material comprises at least about 0.25 percent of each tube section by weight, and more preferably between about 0.05 percent and 1 percent of each tube section by weight. Further, each tube section may have a wall thickness of between about 0.375 in. and 0.50 in. and diameters between about 3 inches and about 9 inches. However, other wall thickness and other diameters are possible and within the scope of the invention. In another approach, the tubing may be coated with the anti-microbial material via a spraying or dipping (bath) process, e.g. with a coating thickness of between about 0.004 in. and 0.020 in., and preferably at least about 0.010 in.

In conjunction with this aspect, transport surfaces (i.e., contact surfaces) within one or more of the user stations, traffic control devices and/or within other pneumatic tubes of the system may comprise an anti-microbial material. For example, one or more of the user stations may include carrier ports with anti-microbial material-containing surfaces. In one approach, such surfaces may be defined by replaceable mats. Such mats may allow for both containing and controlling large quantities of liquid while providing a resiliency to absorb impact forces that may result upon receiving a carrier. Such mats may be manufactured of a material (e.g., a polymeric material) that is blended with the anti-microbial material(s) and/or coated with the anti-microbial material(s).

In a related aspect, an improved pneumatic tube carrier is provided having an interior surface comprising an anti-microbial material as noted above and one or more of the features taught in U.S. Pat. No. 5,980,164, herein incorporated by reference in its entirety. In particular, an improved pneumatic tube carrier may include first and second shell members that are hingedly interconnected and provided with latch means to be selectively openable/closeable. Each of such shell members may be fabricated from a polymeric resin and anti-microbial mixture, wherein the anti-microbial material and its carrier if utilized comprises between about 0.25 percent and about 5 percent of each shell member by weight and more preferably between about 0.5 percent and about 1.0 percent of each shall member by weight. Alternatively, the first and second shell members may each comprise a base structure (e.g. a plastic body) with a coating applied thereto, wherein the coating comprises between about 0.25 percent and about 5 percent in of an anti-microbial material and its carrier if utilized by weight.

To allow the first and second shell members to contain, for example, liquids therein in a close position, the carrier may further include a seal that is disposed between at least a portion of the mating surfaces of the first and second shell members. This seal may be a polymeric material (e.g., a rubberized material) and may further comprise an antimicrobial material.

According to another aspect of the present invention, a carrier for use in a pneumatic transport system is provided that includes at least one wall formed of a polymeric material. Disposed within the matrix of the polymeric material are antimicrobial particles that inhibit microbial growth on at least an inside surface of the carrier. The antimicrobial particles and the polymeric material define a composite sidewall which has an impact strength that is greater than the impact strength of the polymeric material alone. In this regard, the antimicrobial particles form a reinforcement for the polymeric material in addition to providing antimicrobial functionality.

To provide reinforcement to the polymeric material, the concentration of the antimicrobial particles may be less than about 5 percent by weight and more preferably less than about 2 percent by weight of the composite sidewall. Further, individual antimicrobial particles having a maximum dimension of less than about 20 microns may also provide improved mechanical characteristics for the composite sidewall. In one arrangement in the individual antimicrobial particles may be substantially free of rough surfaces to reduce stress concentrations within the matrix of the polymeric material. In this arrangement the antimicrobial particles may comprise glass beads which include antimicrobial metal ions.

As a further optional feature, any of the above noted improved carriers may include a resilient end, or bumper member, at one or both ends thereof. Such a bumper member may be provided to absorb impact forces resulting from the travel of the carrier through a pneumatic carrier system. Such a bumper member may be either permanently affixed to the carrier via a molding process or via secondary mounting process. For example, the bumper member may be temporarily affixed utilizing a tongue and groove arrangement, or, simply mounted to the carrier with screws and/or bolts. Typically, the bumper member will be formed of a durable material and may also include anti-microbial material(s). Further, it may be desirable that the bumper member not significantly modify the overall carrier length. In one application the bumper member is about 0.25 inches thick, but may be between about 0.1 and 0.5 inches thick. This may allow for retrofitting such bumper members onto existing carriers. In any case, the intention of the bumper member is to absorb between about 2 percent and about 20 percent of the energy dissipated during carrier landing.

Additional aspects and advantages of the present invention will be apparent to those skilled in the art upon consideration of the further description that follows.

### BRIEF DESCRIPTION OF THE DRAWINGS

FIG. 1 is a schematic mechanical diagram of a pneumatic tube carrier system.

FIG. 2 is a schematic electrical diagram of the pneumatic tube carrier system of FIG. 1.

FIGS. 3a and 3b are front views of one embodiment of a pneumatic tube system carrier in an opened and closed state, respectively, said carrier being employable in the pneumatic tube carrier system of FIG. 1.

FIG. 3c is a cross-sectional view of a sidewall surface of the pneumatic tube system carrier of FIG. 3a.

FIG. 4 is a cross-sectional end view of another embodiment of a pneumatic tube system carrier employable in the pneumatic tube system of FIG. 1.

FIG. 5a is a perspective cutaway view of the carrier of FIGS. 3a and 3b located within an exemplary pneumatic tube employable in the pneumatic tube carrier system of FIG. 1.

FIG. 5b is a cross-sectional view of an exemplary pneumatic tube employable in the pneumatic tube carrier system of FIG. 1.

FIG. 6 is a top cutaway view of a multi-linear transfer unit (MTU) employable in the pneumatic tube system of FIG. 1.

FIG. 7 is a perspective view of a user station employable in the pneumatic tube carrier system of FIG. 1.

### DETAILED DESCRIPTION OF THE INVENTION

Reference will now be made to the accompanying drawings, which assist in illustrating the various pertinent features of the present invention. Although the present invention will now be described primarily in conjunction with use in a pneumatic tube transport system, it should be expressly understood that the present invention may be applicable to other transport systems. In this regard, the following description is presented for purposes of illustration and description. Furthermore, the description is not intended to limit the invention to the form disclosed herein. Consequently, variations and modifications commensurate with the following teachings, and skill and knowledge of the relevant art, are within the scope of the present invention. The embodiments described herein are further intended to explain best modes known of practicing the invention and to enable others skilled in the art to utilize the invention in such, or other embodiments and with various modifications required by the particular application(s) or use(s) of the present invention.

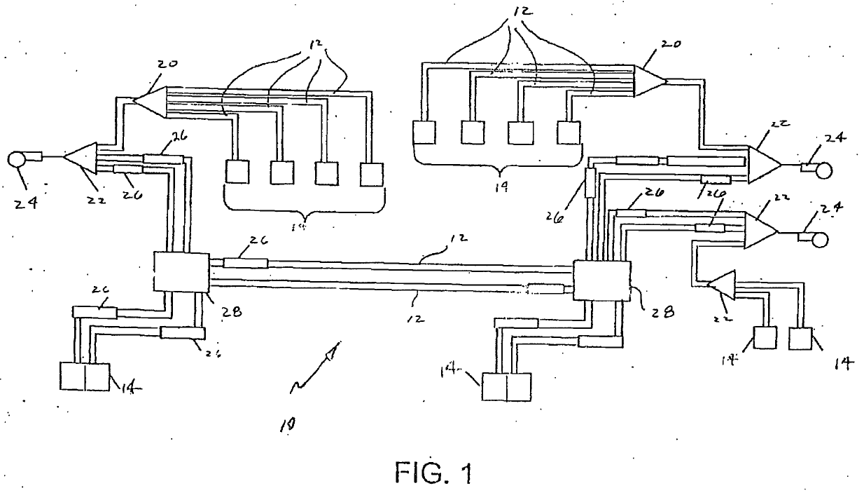

FIG. 1 is a schematic mechanical diagram of a pneumatic tube carrier system 10 within which the present invention is employable. In general, the pneumatic tube carrier system 10 transports pneumatic carriers through pneumatic tubes 12 between various user stations 14, each such transport operation being referred to herein as a “transaction”. At each of the user stations 14, a user may insert a carrier, select/enter a destination address/identification and a transaction priority, and then send the carrier. The system determines an optimum path to route the carrier through the system.

Interconnected with each user station 14 is a transfer unit 20 which orders carriers arriving through different ones of the pneumatic tubes 12 from different stations 14 into a single pneumatic tube. This pneumatic tube is further in connection with a vacuum bypass transfer unit 22 and a blower 24 that provides the driving pneumatic force for carrier movement. A set of one or more transfer units 20, a blower 24 and one or more user stations 14 typically define a single zone, with the corresponding vacuum bypass transfer unit 22 being the point of connection to a network and other zones connected thereto.

Within the network itself, one or more additional traffic control devices are employable for ordering, storing and routing carriers to their selected destinations. One type of device is a traffic control unit (TCU) 26 which is employable to receive, temporarily store and release a number of carriers. In certain configurations, one or more TCUs 26 may be provided to operate as linear storage devices, e.g. on a first in first out (FIFO) basis or last in first out (LIFO) basis. In other configurations, one or more TCUs 26 may be provided to operate as matrix style storage devices which store carriers in two-dimensional matrixes, wherein each carrier is separately storable, retrievable and releasable without movement of other carriers stored in the matrix. In any configuration, the TCU 26 will typically include a receipt mechanism, which provides for the entry of carriers 40 into a storage portion and an exit mechanism, which provides for the release of such carriers 40 from the TCU 26. The TCUs may further include transfer mechanisms (e.g., conveyor belts) for transferring received carriers 40 between storage and the exiting mechanism.

Also included in the network are multi-linear transfer units (MTUs) 28 which have functionality to direct carriers from one pneumatic tube to another. For example, an MTU 28 may receive a carrier released by a TCU 26 in a first tube and direct the carrier along a second pneumatic tube in the system to complete a given transaction.

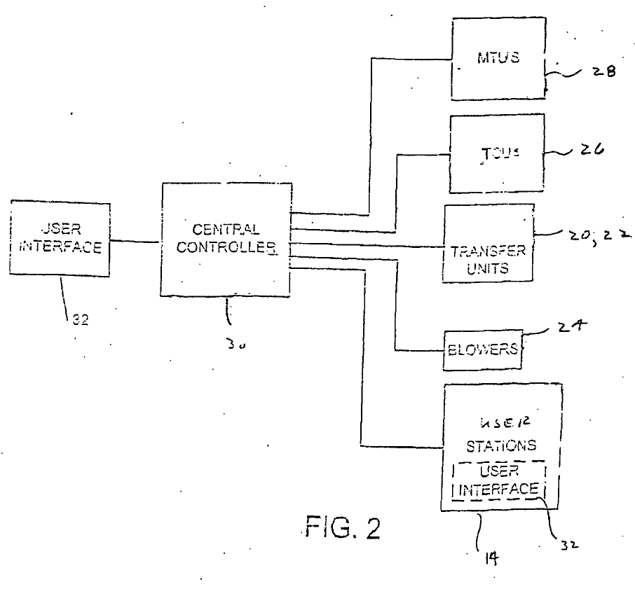

FIG. 2 is schematic electrical diagram for the pneumatic tube carrier system 10. Providing centralized control is a system central controller (SCC) 30. The SCC 30 may include a digital processor and memory. For example, SCC 30 may be configured as one or more programmable digital computers. Connectable to the SCC 30 may be one or more user interfaces 32 through which a system user may monitor the operations of the system and/or manually enter one or more commands to control its operation. Typically, at least one user interface 32 is located at or within an area serviced by stations 14. For example, in a medical facility application, one or more user station 14 and at least one user interface 32 may be provided within each emergency room, laboratory, nursing station, etc. Further, one more user interface 32 may be located at various system control and maintenance locations.

Each of the components 14, 20, 22, 24, 26 and 28 described above in relation to FIG. 1 may include one or more electrical and/or electro-mechanical components which provide for the physical movement of a carrier within the system (e.g. via control of the traffic control devices) and/or the obtainment/provision of information relating to the location of the carriers within the system. In this regard, the noted components shown in FIG. 2 are representations of the various electrical and electromechanical systems that may be employed by the pneumatic carrier system 10. Although in FIG. 2 such components are grouped by type into single blocks, one skilled in the art will realize that the block for each type of device represents the electronics for a number of the same or similar type of components positioned throughout the system.

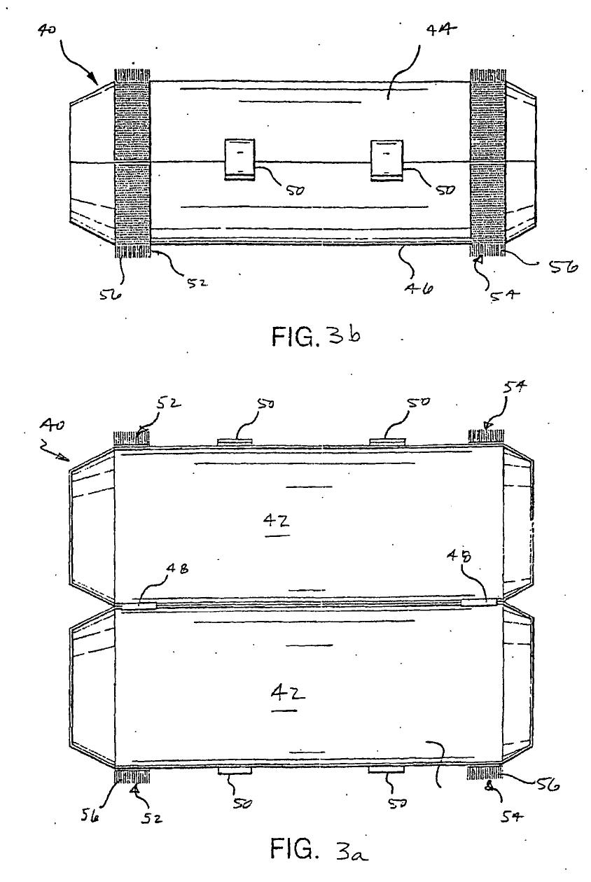

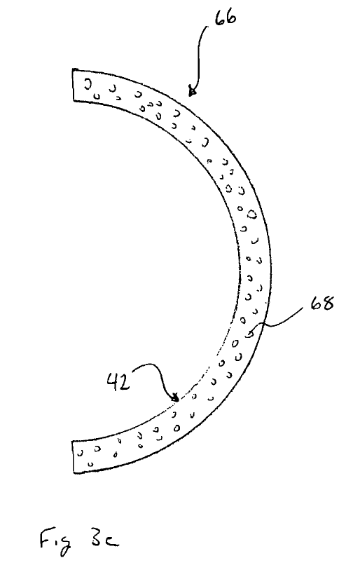

FIGS. 3a and 3b are views of a pneumatic tube system carrier 40 having at least an internal surface 42 comprising an anti-microbial material. The carrier 40 includes first and second shell members 44, 46 that are pivotably interconnected by a hinge member 48. Latches 50 may be provided for securing the first shell member 44 to the second shell member 46 in a closed configuration. Also included as part of the carrier 40 are wear bands 52, 54 for supportably engaging the interior surfaces of pneumatic tubes 12 during transport.

In one embodiment, the first and second shell members 44, 46 each comprise a polymeric resin (e.g., polyethylene, polycarbonate or polyurethane) and an inorganic anti-microbial material, (e.g., glass particles/beads containing silver ions). FIG. 3C shows a cross-sectional view of a sidewall 66 (not to scale) of one of the shell members 44, 46 of the carrier 40. As shown, a plurality of individual antimicrobial particles 68 is disposed within the sidewall 66. That is, the antimicrobial particles 68 may be mixed with the polymeric resin prior to molding of the shell members 44, 46.

The polymeric resin and the antimicrobial particles 68 define a composite sidewall 66. As may be appreciated, the composite sidewall 66 may have improved physical characteristics in comparison to a sidewall formed solely of the polymeric material. For instance, the antimicrobial particles 68 may act as a reinforcement to the polymer matrix thereby improving the mechanical characteristics of the resulting carrier 40. Specifically, it has been found that the impact strength of carriers utilizing composite sidewall greater than the impact strength of carriers that do not include the antimicrobial particles 68. However, if the concentration of the antimicrobial particles 68 exceeds a threshold value and/or if the size of the individual particles is too large, the particles 68 may act to weaken the resulting sidewall structure and, hence, the resulting carrier. For strength purposes, it may also be preferable that the antimicrobial particles have a smooth outer surface to reduce stress concentrations within the polymer matrix. One type of antimicrobial particles that include a smooth outside surface are glass beads containing silver metal ions. Such an anti-microbial material is commercially available from Ishizuka Glass under the tradename IONPURE®.

In any case, a minimum amount of antimicrobial particles is required to provide antimicrobial functionality for the inside surface 42 of the carrier 40. To provide antimicrobial functionality for the inside surface 42 of the carrier 40, the anti-microbial material may comprise between about 0.3 percent and about 5.0 percent of the first and second shell members 44, 46 by weight, and most preferably between about 0.5 percent and about 1.0 percent by weight. In the latter range, the impact strength of carrier 40 may in some instances be increased. The size of the anti-microbial particles may be less than about 40 microns and more preferably less than about 20 microns. Further, the shell members 44, 46 may have a wall thickness of between about 0.090 in. and 0.260 in. and preferably at least around about 0.120 in. Generally, the concentration, type, particle size of the antimicrobial material and/or sidewall thickness of carrier and polymeric resin material, may be adjusted/selected to produce a carrier having one or more desired mechanical characteristics. For instance, it may be desirable that the sidewall of a resulting carrier 40 have an impact strength of at least about 6-8 thousand psi.

One arrangement of the various carrier properties may advantageously yield an at least partially translucent carrier 40. This may allow a system user to distinguish whether or not a carrier 40 has a payload at, for example, a distance of 2-3 feet from the station 14. Producing an at least partially translucent carrier 44 requires a balance between the polymeric material utilized to form the shells 44, 46 of the carrier 40, the sidewall thickness, the concentration of the antimicrobial particles 68 within the sidewall, and the size and type of antimicrobial particles 68 utilized. To produce an at least partially translucent antimicrobial carrier the base polymeric resin may be a substantially translucent material (e.g., having a transmittance/translucency of greater than about 50 percent and more preferably greater than about 80 percent). Generally, it has been found that concentrations of less than 1.0 percent by weight of an antimicrobial material in the base polymeric resin results improved translucency. Further, it has been found that use of smaller anti-microbial material particles 68 result in an increased clarity/translucency of the carrier 40. Finally, it has been found at use of an antimicrobial material that is: incorporated in an at least partially translucent carrier (e.g., glass-beads) further improves the translucency of the resulting carrier 40. The translucency is measured in how much light per 0.100 inches of material the light must pass. In one particular embodiment, an at least partially translucent carrier is formed from a substantially clear polycarbonate (e.g., transmittance of around 88 percent) having 0.5 percent by weight of 20 micron glass beads containing silver metal ions. The carrier 40 has a wall thickness of about 0.120 inches. It has been found that the carrier 40 with these material properties provides antimicrobial functionality while maintaining desired mechanical properties. Specifically, the translucency of the carrier allows a user to ascertain if contents are present within the carrier. In order to improve the ability to ascertain the presence of contents of within the translucent carrier 40, the receiving bin of the station 14 may also be back-lighted, as will be more fully discussed herein.

Though it may be desirable in some instances to have an at least partially translucent carrier, colored features may be included. That is, one half (or all) the carrier 40 may be color coated and/or formed out of a colored (e.g., opaque) polymeric material to distinguish between carriers 40. Further, the carrier wearbands 52 may employ different colors to distinguish between carriers 40, while maintaining maximum viewing of the payload. In other cases, the carrier 40 may be labeled utilizing an adhesive backed media, such that the user may create a carrier disruption upon dispatch of said carrier 40. Further, preprinted labels may be supplied from the factory and installed upon receipt of carriers 40 to distinguish between carriers 40.

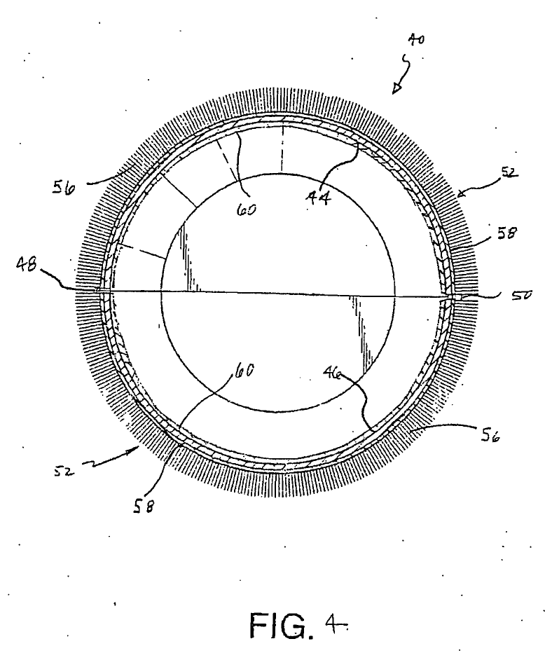

In another embodiment shown in FIG. 4, the internal surface 42 of pneumatic tube carrier 40 may be defined by a coating layer 60 applied to the first and second shell members 44, 46. In this embodiment, the coating layer 60 contains an inorganic anti-microbial material. For instance the coating layer may comprise a polymer resin that includes, for example, zeolite particles containing silver metal ions. Such an anti-microbial material is commercially available from AglON Technologies, Inc., of 60 Audubon Road, Wakefield Mass. 01880. Other antimicrobial materials may be used as well. The anti-microbial coating material may comprise at least about 0.03 percent of the coating by layer weight, and preferably between about 0.05 percent and an about 5.0 percent of the coating layer by weight. Further, the coating layer 60 may have a thickness of at least about 0.010 in. and preferably between about 0.004 in. and 0.020 in.

The first and second wear bands 52 and 54, may each include a plurality of fibers 56 which are mounted upon a backing strip 58. In one embodiment, the fibers 56 are oriented substantially perpendicular to the backing strip 58, wherein adjacent ones of the fibers are oriented substantially parallel to each other. Each of the first and second wear bands 52, 54 may have a packing density of fibers 56 of at least about 4000 fibers per square inch and more preferably between about 10,000 and 100,000 fibers per square inch. Relatedly, each fiber 56 may have an outside diameter of less than about 0.010 inches and more preferably have an outside diameter between about 0.003 inches and about 0.0005 inches.

Fibers 56 may comprise an anti-microbial material mixed with a resin material from which the fibers 56 are formed, wherein the anti-microbial material comprises at least about 0.03 percent of each of the fibers 56 by weight. In another embodiment, the fibers 56 may each have an anti-microbial coating applied thereto, wherein the coating layer comprises at least about 0.03 percent of an anti-microbial material by weight. Further, an anti-microbial fiber may be dipped into a polymer substance to coat the fiber with a durable resin while maintaining an anti-microbial core. The anti-microbial may then permeate the durable resin and provides an anti-microbial surface. In addition to dip coating, the durable resin may be applied via chemical transference, physical transference or any other suitable means. Alternatively, the anti-microbial material may be coated or mixed into the base material of the wear bands 52 and 54 prior to integrating the fibers 56 therein.

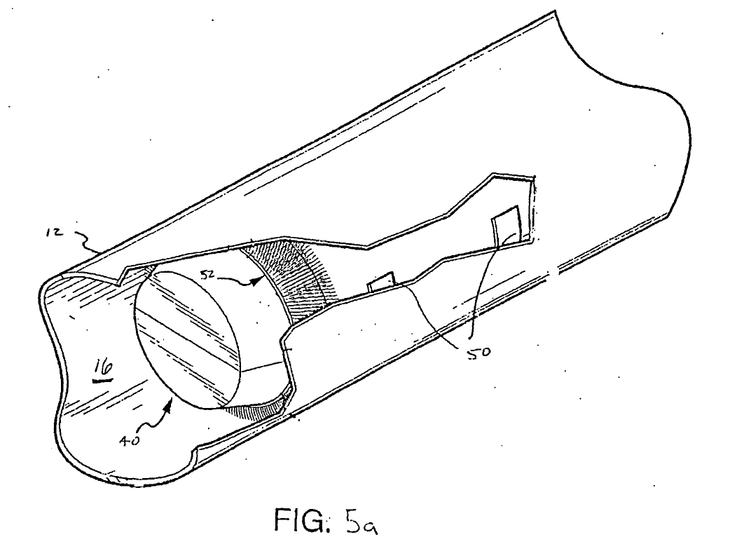

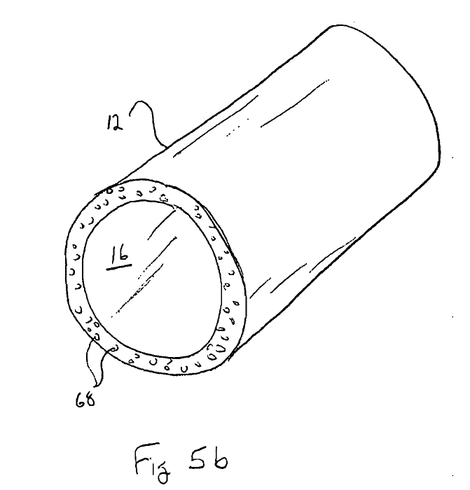

As may be appreciated, in addition to the various componentry of pneumatic tube carrier 40, other components of the system 10 may advantageously comprise carrier contact surfaces containing an anti-microbial material. That is, such contact surfaces may be coated with an anti-microbial material, or, such contact surfaces may be formed with antimicrobial materials within their structure. For example, and with reference to FIG. 5a, the internal surface 16 of one or more of the pneumatic tubes 12 may each comprise an anti-microbial material coating. Alternatively, the tubes 12 may be formed from a polymeric resin (e.g., polyvinylchloride or ‘PVC’, polycarbonate, acrylic, etc.). As with the polymeric carriers discussed above, antimicrobial particles 68 may be incorporated into the sidewall of the tubes 12 when the tubes 12 are formed. See FIG. 5b. As with the polymeric carriers, the concentrations, size and type of the antimicrobial particles may be selected to provide one or more desired properties for the resulting tube 12.

In other embodiments, transfer units 20 (or diverters), MTU's 28, storage tubes, and/or other system components having carrier contact surfaces may comprise such anti-microbial material(s). Such carrier contact surfaces may comprise rigid surfaces as well as flexible surfaces such as conveyor belts. Accordingly, the composition and/or application of the anti-microbial material may be altered for a given application. For instance, anti-microbial materials may be incorporated into the matrix of some components and applied to the surface of other components.

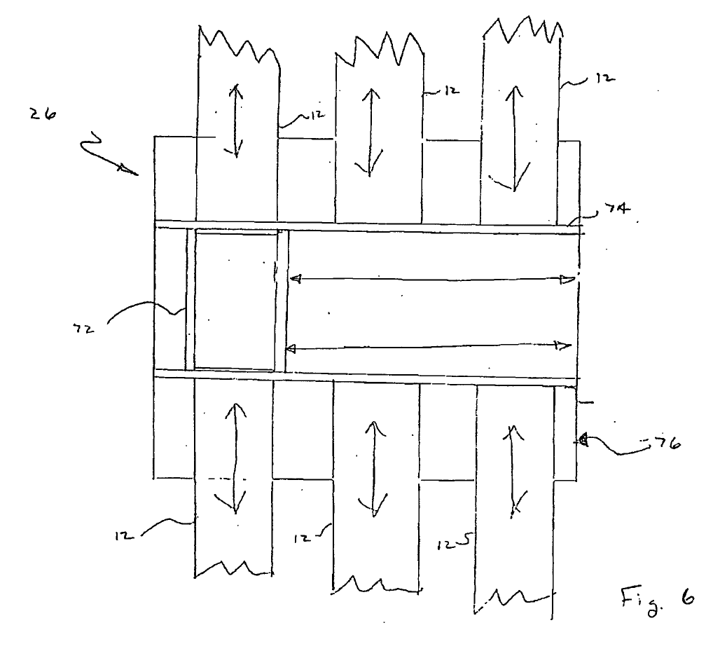

FIG. 6 is a breakaway view of an exemplary MTU 28 embodiment. As can be seen, the MTU 28 is interconnected with a number of incoming pneumatic tubes 12 through which carriers 40 are delivered to the MTU 28. Exiting from the MTU 28 are a number of exit tubes 12 which direct a carrier 40 to a destination zone. Included in the MTU 28 is a carrier delivery device 70 or ‘bucket’ that is moveable along guides 74 and 76 so as to receive carriers 40 directed to the MTU 28 through pneumatic tubes 12, and in response to an instruction signal received from the central controller 30, move the received carrier 40 along the guides 74 and 76 to align the carrier 40 with a selected exit-pneumatic tube 12. Once a pneumatic vacuum force is applied to the selected exit tube 12, the bucket 72 releases the carrier 40. To reduce microbes present in the overall system 10, the internal contact surfaces of carrier delivery device 72 may comprise an anti-microbial material.

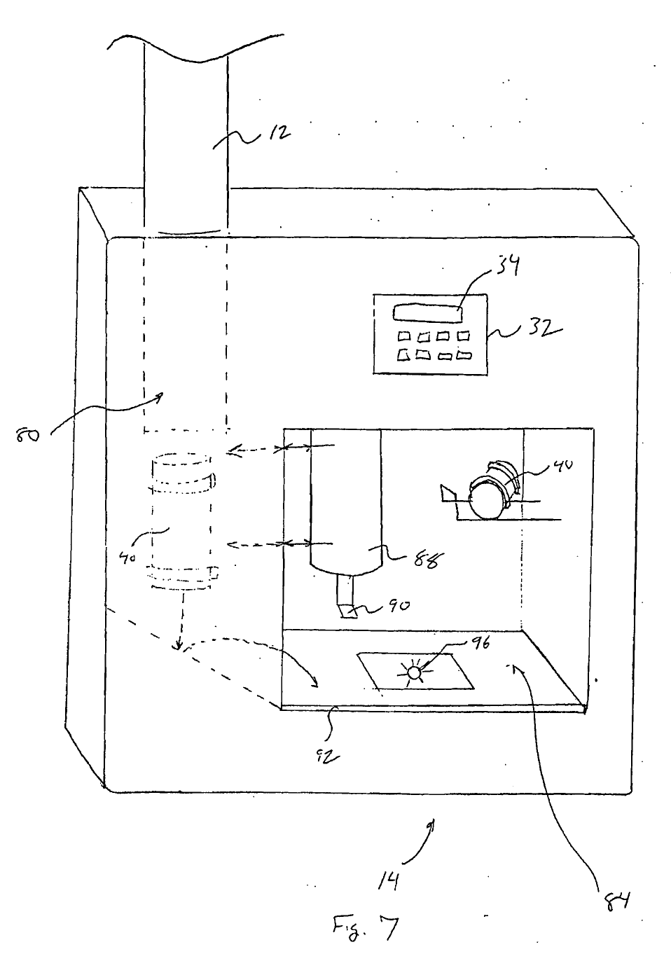

FIG. 7 shows an exemplary view of a user station 14 that is employable in the pneumatic carrier system described herein. As shown, the station 14 includes a sending/receiving port 80 for sending/receiving a carrier 40 to/from pneumatic tube 12. Also included with the station 14 is a user interface 32 which includes a number of interactive devices which a system user may employ for entering information including, for example, destination, priority and security information (e.g., a station identification number 10) for sending a given carrier 40 through the system. The user interface 32 is also employable for entering information for receiving a carrier 40 at a station 14. For example, if a carrier 40 has security information associated with it, this information can be entered into the user interface 32 to complete delivery of the carrier 40 to the destination location. Also included with the user interface is a display 34 which is configured to present messages relating to transaction and system status which are viewable by a system user.

As shown in FIG. 7, the port 80 comprises a discharge tube extending from the pneumatic tube-into the user station 14. Upon receiving a carrier 40, the carrier descends into the user station 14, passes through the port 80 and falls into the receiving bin 84. The user station 14 further includes a dispatcher 88 sized to hold a carrier 40 and which may be disposed toward and away from the receiving port 80. Such movement may be initiated by a system user (e.g., by hand). As shown, the dispatcher 88 comprises a tube into which a carrier 40 is placed and further includes a retainer 90 that engages an end surface of the carrier 40 prior to sending the carrier 40 through the system. Once the carrier 40 is disposed in the dispatcher 88, the dispatcher 88 may be disposed beneath the receiving port 80 to transfer the carrier 40 into the pneumatic tube 12. As will be appreciated, the station 14 may further comprise a belt system (not shown) for initiating loading of the carrier 40 into the pneumatic tube 14. Further, once the carrier 40 is launched into the pneumatic tube 12, the dispatcher 88 may be moved away from the receiving port 80 such that additional carriers may be received from the system without interference. Of note, the exposed surfaces of the dispatcher 88 and the port 80 that contact each given carrier 40 may comprise an anti-microbial material.

As noted, carriers 40 received at the interface 14 drop through the receiving port 80 and fall into the receiving bin 84. In this regard, the bottom of the receiving bin 84 may be a compliant material (e.g., a mesh or sling) to reduce impact. Alternatively, the bottom of the receiving bin 84 may be coated or otherwise covered to reduce impact. By way of example, the bottom of the receiving bin 84 may be defined by a selectively positionable mat 92 that may be replaced as needed. In this regard, the mat 92 may comprise a polymer material or other appropriate material into which an anti-microbial material may be mixed.

As noted above, it may be, in some instances, desirable to determine whether the carriers 40 include a payload. In this regard, it may be desirable to incorporate a backlighting system into the user interface such that a system user may more easily determine whether the typically translucent carriers 40 include such a payload. As shown in FIG. 7, a backlight 96 is incorporated into the bottom of the receiving bin 84 such that light may project through carriers 40 disposed on the bottom surface of the receiving bin 84. When incorporating a mat 92 into the receiving bin 84, utilization of such a backlight 96 may require that the mat 92 include an aperture/window 94 to permit passage. Accordingly, such a window may also incorporate anti-microbial materials. Further, backlighting may be incorporated into the back wall of the receiving bin 84 as well. Irrespective of the exact configuration of the interface 14, all of the contact surfaces of the user interface that contacts the carriers 40 may comprise an anti-microbial material.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A carrier for use in a tube transport system comprising a first tube, said carrier comprising:
a containment vessel having a sidewall defining an enclosed space and which includes a first access to said enclosed space; and
an antimicrobial material associated with at least an inside surface of said sidewall for inhibiting microbial growth on said inside surface of said sidewall.

2. The carrier as claimed in claim 1, wherein said antimicrobial material comprises a plurality of antimicrobial particles including:
a carrier material; and
at least a first inorganic antimicrobial material.

3. The carrier as claimed in claim 2, wherein said inorganic antimicrobial material comprises a metal.

4. The carrier as claimed in claim 3, wherein said inorganic antimicrobial metal comprises silver.

5. The carrier as claimed in claim 2, wherein said carrier material comprises an inorganic non-metallic material.

6. The carrier as claimed in claim 2 wherein said carrier material comprises a ceramic material.

7. The carrier as claimed in claim 6, wherein said ceramic material comprises a silicate material.

8. The carrier as claimed in claim 7, wherein said silicate material comprises a glass.

9. The carrier as claimed in claim 1, wherein said antimicrobial material comprises a coating applied to said inside surface.

10. The carrier as claimed in claim 2, wherein said antimicrobial particle is disposed within a matrix of said sidewall.

11. The carrier as claimed in claim 10, wherein said sidewall is formed of a polymeric material.

12. The carrier as claimed in claim 10, wherein said antimicrobial particles comprise between about 0.25 percent and about 5.0 percent by weight of said sidewall.

13. The carrier as claimed in claim 12, wherein said antimicrobial particles comprises between about 0.5 percent and about 1.0 percent by weight of said sidewall.

14. The carrier as claimed in claim 10, wherein a size of individual antimicrobial particles disposed in said sidewall is less than about 40 microns.

15. The carrier as claimed in claim 10, wherein a size of individual antimicrobial particles disposed in said sidewall is less than about 20 microns.

16. The carrier as claimed in claim 1, wherein at least a portion of said sidewall is translucent.

17. The carrier as claimed in claim 1, wherein first access to said enclosed space further comprises a latch on an outside surface of said carrier for securing said first access in a closed position, wherein said latch further comprises an antimicrobial material.

18. The carrier as claimed in claim 1, further comprising:
at least one wear band interconnected with an outer surface of said containment vessel, wherein said wear band further comprises an antimicrobial material.

19. A tube transport system for transporting a pneumatic carrier between a first location and a second location, the system comprising;
at least a first tube pneumatically interconnecting said first and second locations, said first tube including an inner surface and an outer surface, wherein said inner surface defines a bore sized to receive a pneumatic carrier;
an inorganic antimicrobial material associated with at least said inside surface of said first tube for inhibiting microbial growth on said inside surface of said first tube.

20. The tube system of claim 19, wherein said tube is a metal tube.

21. The tube system of claim 19, wherein said antimicrobial material comprises an antimicrobial coating applied to said inside surface.

22. The tube system of claim 19, wherein said tube is formed of a polymeric material.

23. The tube system of claim 22, wherein said antimicrobial material is disposed within a matrix of said polymeric material.

24. The tube system of claim 19, wherein said antimicrobial material comprises a plurality of antimicrobial particles including:
a carrier material; and
at least a first type of an inorganic antimicrobial material.

25. The tube system of claim 24, wherein said inorganic antimicrobial material is a metal ion.

26. The tube system of claim 25, wherein said metal ion comprises a silver ion.

27. The tube system of claim 24, wherein said carrier material comprises at least one of:
ceramic; and
glass.

28. The tube system of claim 19, wherein a diameter of said tube is between about 10 cm and about 16 cm.

29. The tube system of claim 19, further comprising:
a transfer unit for transferring a pneumatic carrier from said first tube to a second tube, wherein at least a first contact surface of said transfer unit that contacts the pneumatic carrier comprises an antimicrobial material.

30. The tube system of claim 19, further comprising:
at least a first user station, said user station being operative to send and receive said pneumatic carriers, wherein at least a first contact surface of said user station that contacts the pneumatic carrier comprises an antimicrobial material.

31. A carrier for use in a tube transport system comprising a first tube, said carrier comprising:
first and second shells for engagement one with the other in a closed position, said first and second shells defining an enclosed space in said closed position;
hinge means coupled to the first and second shells, said hinge adapted to permit movement of the first and second shells between said closed position and an open position;
an antimicrobial material associated with at least an inside surface of said first and second shells for inhibiting microbial growth on said inside surface of said first and second shells.

32. The carrier as claimed in claim 31, further comprising:
a seal situated between at least a portion of contacting of said first and second shells for forming a liquid barrier when said first and second shells are in said closed position, wherein said seal comprises an antimicrobial material.

33. The carrier as claimed in claim 31, further comprising:
a latch for maintaining said first and second shells in said closed position, wherein said latch comprises an antimicrobial material.

34. The carrier as claimed in claim 31, further comprising:
at least one seal band interconnected with an outer surface of at least one of said first and second shells, wherein said seal band further comprises an antimicrobial material.

35. The carrier as claimed in claim 31, wherein said first shell is at least partially transparent and said second shell is opaque.

36. The carrier as claimed in claim 31, wherein said first and second shells comprise a polymeric material and said antimicrobial material is disposed within a matrix of said polymeric material.

37. A carrier defining an accessible enclosed space for use in a tube transport system comprising a first tube, said carrier comprising:
at least one wall comprising a polymeric material having a plurality of antimicrobial particles disposed within a matrix of said polymeric material, wherein said antimicrobial particles inhibit microbial growth on a surface of said wall; and
wherein said polymeric material with said antimicrobial particles disposed within said matrix defines a composite material, wherein an impact strength of said composite material is greater than an impact strength of said polymeric material.

39. The carrier as claimed in claim 37, wherein said wall is at least partially transparent.

40. The carrier as claimed in claim 37, wherein said antimicrobial particles comprises a plurality of individual particles each having a maximum dimension of less than about 20 microns.

41. The carrier as claimed in claim 40, wherein an outside surface of said individual particles is substantially free of rough edges.

42. The carrier as claimed in claim 37, wherein said antimicrobial material comprises between about 0.25 percent and about 2.0 percent by weight of said composite material.

43. The carrier as claimed in claim 37, wherein said antimicrobial material comprises:
a carrier material; and
at least a first inorganic antimicrobial material.
